(19) United States
(12) Patent Application Publication
(10) Pub. No.: US 2024/0218176 A1
(43) Pub. Date: Jul. 4, 2024

(54) MOLDABLE, HYDROPHILIC POLYMER BLEND FOR MOLDING DEVICES WHICH ARE HYDROPHILIC AND LUBRICIOUS AFTER MOLDING

(71) Applicant: JONSMAN INNOVATION ApS, Gørløse (DK)

(72) Inventor: Jacques Jonsman, Gørløse (DK)

(73) Assignee: JONSMAN INNOVATION ApS, Gørløse (DK)

(21) Appl. No.: 18/558,438

(22) PCT Filed: May 3, 2022
(86) PCT No.: PCT/EP2022/061880

§ 371 Date: Nov. 1, 2023

(30) Foreign Application Priority Data
May 3, 2021 (DK) PA202100453

(51) Int. Cl. C08L 71/02

(52) U.S. Cl. CPC C08L 71/02; C08L 2203/02

(57) ABSTRACT

For providing molded polymer articles having surfaces when wet which are lubricious after molding, there is proposed a polymer blend comprising a matrix polymer and a hydrophilic polymer composition comprising polyethylene-oxide; said matrix polymer selected from a TPE or a TPU matrix polymer presenting a flat-surface Shore A hardness from 40 to 75 according to ISO 48-4:2018; said polymer blend comprising, by total polymer blend weight, from 20 w/w % to 65 w/w % of said hydrophilic polymer composition with the balance matrix polymer; said hydrophilic polymer composition comprising, by weight of said hydrophilic polymer composition, from 5 w/w % to 30 w/w % of a PEG-plasticizer having a molecular weight from 200 D to 600 D, and polyethyleneoxide having a molecular weight from 100,000 D to 750,000 D as balance, uses thereof and molded articles therefrom.

### TECHNICAL FIELD

In the field of moldable medical devices there is suggested polymer compositions and uses of such compositions, which compositions are moldable into medical devices and being hydrophilic and lubricious after molding.

### BACKGROUND

Within the field of medical devices, wherein the device or a part of the device is in direct contact with, e.g., a body cavity, blood vessel, or such like, e.g., a catheter for insertion into the body, it has often been found necessary in the art to provide the surfaces of such medical devices having direct contact with e.g., a body cavity, lubricious and/or hydrophilic surfaces for minimizing the potential for contact damage to the body tissue from the contact between the medical device and the body, c.f. e.g., WO 2006/032043. By providing contact surfaces adapted for being friction reducing, e.g., lubricious and/or hydrophilic, the reduced friction and/or increased hydrophilicity and associated hydration layer formed between the device and the body, patient discomfort, experienced pain and/or the risk of tissue and/or device damage by contact is mitigated, at least in part, by the increased compatibility between the device and the body tissue.

In the current art, medical devices requiring a lubricious surface are usually in a first step molded in a polymer material suitable for providing the necessary structural properties to the medical device and subsequently in a second step, e.g., when a lubricious and/or hydrophilic surface is desired, the device is provided with a coating layer having the desired lubricious and/or hydrophilic properties, e.g., by deposition of the lubricious coating layer on the formed medical device.

While less common, in the art it is also known to co-mold the device with a structural polymer and a lubricious polymer coating layer, c.f. e.g., U.S. Pat. No. 5,084,315.

In WO 2019/099080, pertaining to the general background art of the present application, is disclosed a material for making a stent comprising a soft material, which is a thermoplastic polyurethane, and a hard material, which may be a polyethylene glycol, such as e.g., polyethylene oxide. The stent composition comprises 3-20 wt % hard material and soft material, based on the total weight of the stent, wherein the mixed material has a hardness of from Shore A55 to Shore A90 and wherein the stent is made by extruding a stent precursor thread followed by 3D printing.

Usually, however, a polymer coating layer suitable for use as a lubricious surface on the medical device is not lubricious directly after co-molding or deposition, but only after activation. In many situations, the coatings comprise polymers which are not per se hydrophilic polymers, but which polymers rather are partially soluble in water, wherein the coating upon water contact, and, typically, swelling with water produces a low friction hydrogel on the device surface, which low friction hydrogel provides the actual lubricious coating.

Such lubricious, hydrophilic coatings generally work well; however, the coating process is expensive and there are several unfortunate error/failure modes of the coating and coating process.

For these reasons, a polymer material that does not need be provided a hydrophilic coating but have hydrophilic properties intrinsically after a device is molded or extruded in the aforesaid polymer material is sought. Such a material will significantly simplify production of medical device, reduce costs, and eliminate or reduce known error modes of production.

It is well known that mixing a matrix polymer with a hydrophilic polymer will give a co-polymer material which becomes lubricious when wetted, c.f. e.g., U.S. Pat. No. 5,084,315. Herein is taught in a preferred example to prepare a polymer coating layer which becomes lubricious after wetting comprising a compound polymer comprising a lubricating polymer suitable for providing lubricity to a coated article when wet, a matrix polymer, an optionally a PVC base component. In a preferred embodiment, the matrix polymer is a polyurethane comprising between 20% to 75% by weight hard segment of MDI and BDO, and between 80% to 20% by weight soft segment of PTMEG and PEG, and wherein the lubricating polymer can be a polyethyleneoxide having a molecular mass of between 100 kD to 8,000 kD.

Such polymer blends obtained from compounding (mixing) a matrix polymer with a hydrophilic polymer will give molded and extruded devices having some of the in-mixed hydrophilic polymer located at the surface of the device. When the device contacts liquid water, the hydrophilic polymer dissolves in the water and thereby lubricating the surface.

However, as the hydrophilic polymer is dissolved, such surfaces as known in the art are liable to loss, often rapid loss, of the in-mixed hydrophilic polymer, which is removed from the surface over time by transport in the water phase. Here, the hydrophilic polymer is removed fastest from the surface when the device is in mechanical contact with another surface as is the case when lubrication is relevant.

Accordingly, it is a problem in the art that the device lubriciousness introduced by compounding of matrix polymer and hydrophilic polymer is too short-lived compared to dip-coated surfaces commonly used in medical device in the art.

An associated problem with compounded hydrophilic polymer dissolving out of the matrix polymer is that the molded device loses structural stability, both to the loss of constituent polymers, but also due to solvent intrusion into the molded layer and associated layer expansion.

The present invention relates to polymer compound materials for use in molding medical devices, which are intrinsically lubricious and/or hydrophilic after molding without further activation, further having improved benefits of increased lubricity, activation speed, longevity, and dimensional stability in use.

### DETAILED DESCRIPTION

In a first aspect of the present invention, there is detailed a polymer blend comprising a matrix polymer and a hydrophilic polymer composition comprising polyethylene-oxide; the matrix polymer selected from a TPE or a TPU matrix polymer presenting a flat-surface Shore A hardness from 40 to 75 according to ISO 48-4:2018; the polymer blend comprising, by total polymer blend weight, from 20 w/w % to 65 w/w % of the hydrophilic polymer composition with the balance matrix polymer; the hydrophilic polymer composition comprising, by weight of the hydrophilic polymer composition, from 5 w/w % to 30 w/w % of a PEG-plasticizer having a molecular weight from 200 D to 600 D, and polyethyleneoxide having a molecular weight from 100,000 D to 750,000 D as balance.

In an embodiment thereof there is detailed, the polymer blend according to any previous aspect or embodiment, wherein the flat-surface Shore A hardness is from 45 to 75 according to ISO 48-4:2018.

In an embodiment thereof there is detailed, the polymer blend according to any previous aspect or embodiment, wherein the hydrophilic polymer composition comprises polyethyleneoxide having a molecular weight from 200,000 D to 500,000 D.

In an embodiment thereof there is detailed, the polymer blend according to any previous aspect or embodiment, wherein the polyethyleneoxide has a molecular weight of 400,000 D.

In an embodiment thereof there is detailed, the polymer blend according to any previous aspect or embodiment, wherein the PEG-plasticizer has a molecular weight from 300 D to 500 D.

In an embodiment thereof there is detailed, the polymer blend according to any previous aspect or embodiment, wherein the PEG-plasticizer has a molecular weight of 400 D.

In an embodiment thereof there is detailed, the polymer blend according to any previous aspect or embodiment, wherein the hydrophilic polymer composition comprises, by weight of the hydrophilic polymer composition, from 10 w/w % to 20 w/w % of the PEG-plasticizer.

In an embodiment thereof there is detailed, the polymer blend according to any previous aspect or embodiment, wherein the hydrophilic polymer composition comprises, by weight of the hydrophilic polymer composition, 15 w/w % of the PEG-plasticizer

In an embodiment thereof there is detailed according to any previous aspect or embodiment, the polymer blend comprising a matrix polymer and a hydrophilic polymer composition comprising polyethylene-oxide; the matrix polymer selected from a TPE or a TPU matrix polymer presenting a flat-surface Shore A hardness from 45 to 75 according to ISO 48-4:2018; the polymer blend comprising, by total polymer blend weight, from 20 w/w % to 65 w/w % of the hydrophilic polymer composition with the balance matrix polymer; the hydrophilic polymer composition comprising, by weight of the hydrophilic polymer composition, from 10 w/w % to 20 w/w % of a PEG-plasticizer having a molecular weight from 300 D to 500 D, and polyethyleneoxide having a molecular weight from 200,000 D to 500,000 D as balance.

In an embodiment thereof there is detailed according to any previous aspect or embodiment, the polymer blend comprising a matrix polymer and a hydrophilic polymer composition comprising polyethylene-oxide; the matrix polymer selected from a TPE or a TPU matrix polymer presenting a flat-surface Shore A hardness from 45 to 75 according to ISO 48-4:2018; the polymer blend comprising, by total polymer blend weight, from 20 w/w % to 65 w/w % of the hydrophilic polymer composition with the balance matrix polymer; the hydrophilic polymer composition comprising, by weight of the hydrophilic polymer composition, from 15 w/w % of a PEG-plasticizer having a molecular weight of 400 D, and polyethyleneoxide having a molecular weight of 400,000 D as balance.

In a second aspect of the present invention, there is detailed the use of a polymer blend according to any embodiment detailed herein, for molding a medical article presenting when wet at least one lubricious surface made from the aforementioned polymer blend.

In a third aspect, there is herein detailed a medical article comprising a polymer blend according to any of the embodiments detailed herein, presenting when wet at least one lubricious surface made from the aforementioned polymer blend.

### Examples

### Sample Preparation:

### Sample Polymers:

### Matrix Polymers:

Tests were performed using common thermoplastic matrix polymers, such as polyethylene (PE), polypropylene (PP), acrylic (PMMA), polystyrene (PS) and polycarbonate (PC). These materials were found to be lubricious with an incorporated hydrophilic polymer only for a few seconds and were not further tested.

Further tests were performed using thermoplastic elastomers (TPE's), thermoplastic polyurethanes (TPU's) further to the common thermoplastic polymers mentioned above.

Test were performed using e.g., Estane™ and Elastollan™ thermoplastic polyurethanes, Topas™ E-140 elastomer, and Kreiburg™ thermoplastic elastomers commercially available over a wide range of Shore A hardnesses.

Specific matrix polymers reported herein are listed by type and tradenames in Tables 2 to 6 reported herein.

### Hydrophilic Polymer for Compounding in a Matrix Polymer:

Relevant lubricating hydrophilic polymers for compounding in a matrix polymer were found to be polyethylene oxide (PEO/PEG), polyethyloxazoline (PEOx) and polyvinyl-pyrrolidone (PVP). These polymers are commonly used in lubricating hydrophilic coatings for medical devices.

In the present experiments, however, PVP and PEOx were found to be unsatisfactory and not tested further. PEO, on the contrary, comprised in a matrix polymer of the experiments was found to extrude and mold and was found to be usable in the polymer compositions of the present invention, if the extruded polymer compositions after compounding were kept below 80% relatively humidity, cf. Table 1.

In the experiments reported here in Tables 2 to 6, the hydrophilic polymer is PEO-400,000 plasticized with 15% PEG-400.

For each test, total concentrations of the hydrophilic polymer with plasticizer are given in the tables such that e.g., 50% w/w by total mass hydrophilic polymer listed in the table is equal to 42.5 w/w PEO-400,000 and 7.5% w/w PEG-400 of the total mass of the polymer blend also comprising the matrix polymer as tested. In the tests, only matrix polymer with hydrophilic polymer and plasticizer were present, the matrix polymer in all reported experiments constituting the balance to 100% w/w by total mass.

TABLE 1
Extrusion of PEO plasticized with PEG

| PEO | Plasticizer | Concentration | Temperature |
|---|---|---|---|
| MW [D] | MW [D] | [w/w %] | [° C.] |
| 1,000,000 | 1,000 | 57.4% | 140° C. |
| 1,000,000 | 400 | 39.5% | 160° C. |
| 1,000,000 | 400 | 24.3% | 200° C. |
| 400,000 | 400 | 25.6% | 180° C. |
| 400,000 | 400 | 22.4% | 180° C. |
| 400,000 | 400 | 20.0% | 180° C. |
| 400,000 | 400 | 17.6% | 200° C. |
| 400,000 | 400 | 16.3% | 200° C. |
| 400,000 | 400 | 15.1% | 200°C. |
| 400,000 | 300 | 15.1% | 200° C. |
| 400,000 | 200 | 15.1% | 200° C. |
| 400,000 | 400 | 13.9% | 200° C. |
| 400,000 | 400 | 12.7% | 200° C. |
| 400,000 | 400 | 9.8% | 200° C. |
| 300,000 | 400 | 20.9% | 170° C. |
| 300,000 | 400 | 15.2% | 170° C. |
| 300,000 | 400 | 10.1% | 170° C. |
| 200,000-300,000 | 400 | 20.1% | 140° C. |
| 200,000-300,000 | 400 | 15.0% | 150° C. |
| 200,000-300,000 | 400 | 10.3% | 150° C. |
| 100,000-200,000 | 400 | 22.2% | 75° C. |
| 100,000-200,000 | 400 | 15.3% | 85° C. |
| 100,000-200,000 | 400 | 8.9% | 85° C. |
| 100,000 | 2,000 | 20.0% | 65° C.-75° C. |
| 100,000 | 2,000 | 10.0% | 65° C.-85° C. |
| 100,000 | 400 | 15.0% | 65° C.-75° C. |
| 100,000 | 400 | 8.0% | 65° C.-85° C. |

### Compounding:

Polymer compounding was done in accordance with good manufacturing practices and standards of the thermoplastic forming industry, by extrusion of the constituent polymer components for preparing a homogenous, extruded polymer composition in the form of an extruded strand comprising the aforementioned constituent polymer components, followed by cooling and cutting of the extruded strand into pellets suitable for use in injection molding. It is considered that the skilled person will know how to prepare a homogenous, extruded polymer composition in the form of an extruded strand comprising the aforementioned constituent polymer components on the basis of the common general knowledge of the same skilled person.

Initially, and in accordance with current industry standards, cooling was by water bath submersion. However, as it was observed that the water cooling led to partial dissolution of the hydrophilic polymer components of the resulting extruded polymer blends, water cooling was abandoned in favor of air cooling.

Significantly improved results with respect to lubrication and moldability of the resulting polymer blends was observed, when polymer compounding was performed in the absence of water, including reduced jamming of the pelletizing apparatus.

When air cooling, it is preferable that the air comprises less than 20% rel. humidity, preferably less than 10% rel. humidity for optimal polymer blend performance. The results reported below were performed on extruded polymer blends dried in air at less than 10% rel. humidity for optimal injection molding results.

### Test Sample Injection Molding:

Test samples for use in the below reported experiments were prepared by injection molding in accordance with standard practices in the art.

### Experimental:

### Shore A Hardness Measurements

Shore A hardness was measured on test samples prepared as detailed above using a handheld Rex durometer model 1600 in accordance with the manufacturer's instructions (https://www.durometer.com/wp-content/uploads/REXOpInstrucv1214r.pdf), accessed Apr. 15, 2021) at room temperature.

For the results presented herein, the Shore A-precision provided by a handheld instrument is sufficient for deducting the experimental conclusions, however for determining the scope of protection, the Shore A-hardness of a given sample should be evaluated following ISO 48-4:2018—“Rubber, vulcanized or thermoplastic—Determination of hardness—Part 4: Indentation hardness by durometer method (Shore hardness)”.

Assessment of Test Sample Properties when Wet—Assessment of Lubricity, Durability, Activation Speed, and Dimensional Stability:

In the art, phenomenological characteristics used to describe a hydrophilic lubricating surface comprise lubricity, durability, activation speed and dimensional stability, are well-known in the relevant technical field. Unfortunately, no standards exist for the measurement of these phenomenological characteristics as in general, they are not objectively quantifiable, and test sample/device geometry strongly influences observable characteristics between experiments. Measuring e.g., friction and lubrication on a cylindrical tube (for example a catheter), provides apparent observable test values different from apparent observable test values obtained using e.g., test samples having a flat surface, and again different from surfaces with non-constant curvatures.

For this reason, it is customary in the art and of manufacturers of lubricious medical devices generally use their own internally developed methods of measuring the lubricating characteristics of their devices.

Due to the non-standardized, phenomenological nature of reported lubriciousness characteristics, the results reported herein below are graded on a 0 to 10 scale, where 0 is worst and 10 is best. The scale is internally balanced as a specific scale value is not assigned to a test sample at measurement or test, but only after all samples have been measured or tested, thereby assuring that an early assessment of a specific scale value of one test sample does not contort the grade scale or cause a recalibration of the grade scale.

### Assessment—Lubricity

The reported lubricity is measured as lubrication by a semi-automated custom-built system. A standardized rubber pad is moved back and forth over the sample while it is submerged in water. The lateral force exerted on the rubber pad is recorded for each movement of the rubber pad. The value of the recorded force stands in proportion to the kinematic friction force and is in the presently reported experiments taken as a measure of the lubricity. The tests reported herein are comparable to the tests presented in Example IV of U.S. Pat. No. 5,084,315, with the difference that since no absolute value for the coefficient of friction was attempted to be established, internal friction could be ignored, and a semi-linear scale established for internal comparison as reported in the below tables.

### Assessment—Durability

Using the data recorded under the measurement of lubricity, the lateral force on the rubber pad for each movement at constant velocity is concomitantly measured. The lateral force necessary for achieving constant velocity generally starts low as the prepared samples are at maximum lubriciousness when first exposed to water. Gradually, however, the lateral force necessary to move the rubber pad increases and settles on a higher force dependent on the surface material on which the lubricious layer has been provided. As this higher force can be determined prior to providing a lubricious layer. For practical reasons, the present grade scale is based on counting the number of movements needed for 20% of the maximum lateral force, which for the samples reported below varied between 5 movements to several thousand movements.

### Assessment—Activation Speed

For serving the purpose of a lubricious polymer surface in medical devices, it is important that a surface coated with a polymer coating which is lubricious when wetted, is rapidly activated by wetting such that secondary biological deposits on the coated surfaces are avoided before wetting, and the desired property of lubricity when wetted is rapidly established. In the art, this is known as the process of activation or activation by wetting.

In the experiments reported herein, activation by wetting generally was very fast with an activation velocity below 5 seconds, typically between 1 to 5 seconds. This result is consistent with the prior art expectation, wherein a surface coated with a polymer comprising a matrix polymer embedding a hydrophilic polymer in general is rapidly activated upon wetting and is a desired feat in a successful lubricious polymer coated surface.

Accordingly, in the experiments reported herein, it was impossible to measure an approximated activation velocity.

However, it was discovered that a tactile sensing grading was possible, providing at least a subjective indication of the grade difference between coated surfaces with respect to their activation speeds.

In the tactile sensing method applied, a test sample was submerged in water and the person performing the test immediately pressed a finger against the surface and started to rub the finger over the surface, while timing the time from the finger touching the surface to feeling that the wetted surface lubricity no longer increased. In this manner, a relative grading could be established. For consistency, the grading experiment was done for all test samples and references shortly after each other, for facilitating comparison of the samples.

### Assessment—Dimensional Stability

A general problem of hydrophilic and wettable polymer compositions at surfaces is that when wetted, the surface comprising the polymer composition will absorb water and thereby swell. The swelling affects the geometry of the underlying elements, e.g., a medical device. Generally, the swelling will give rise to expansion, typically linear expansion, of the underlying element in all dimensions, but as some surfaces swell more than others, the underlying element, e.g., a medical device, will thus also warp and twist.

While it is conventional and within the skills of the person in the art to measure linear expansion after a test sample detailed herein is subjected to wetting by submersion in water, warping is difficult to quantify consistently between test samples.

For this reason, the reported grading of the dimensional stability of the reported test samples is based on the measured linear expansion for a given test sample in combination with a subjective evaluation of the warping for a combined grade score.

### Experimental Results

### Optimum Molecular Mass of PEO for Compounding

In experiments reported herein and others performed but not reported, PEO's with molecular masses between 100 kD to 700 kD (kilo-Daltons) were found to provide practical results when compounded with a TPE and TPU matrix polymer, both with and without plasticizer, as will be discussed below. Optimal results were found for PEO's having molecular masses from 300 kD to 500 kD, more optimally from 325 kD to 475 kD, from 350 kD to 450 kD, even more optimally from 375 kD to 425 kD. At a molar mass of PEO of 400 kD as reported herein, and within the optimal ranges reported, the PEO's compounded alone, but preferably compounded with a plasticizer, molded and extruded very well, were found to have good plastic temperature ranges for subsequent injection molding for molding a medical device comprising the polymer compositions detailed herein, while providing very suitable lubricious polymer compounds for the polymer blends of the invention in accordance with the applied grade scale reported herein.

In experiments not otherwise reported, the following PEO/PEG hydrophilic polymers were tested: PEG-200, PEG-300, PEG-400, PEG-600, PEG-1000, PEG-1500, PEG-2000, PEG-6000, PEG-8000, PEG-10000, PEG-20000, PEG-35000, PEG-40000, PEO-100000, PEO-160000, PEO-200000, PEO-250000, PEO-300000, PEO-350000, PEO-400000, PEO-450000, PEO-500000, PEO 700000, PEO-1000000, and PEO-4000000.

For these compounds it was found that polymer compositions comprising PEO with molecular masses less than 50 kD did not compound satisfactorily, and that PEO with molecular masses from 1,000 kD and up likewise did not compound nor mold satisfactorily.

Results for PEO-400000 compounded in respective matrix polymers in the presence of a plasticizer are reported herein, Table 1.

TABLE 2

| | | Material | Measured | Hydrophilic | Lubricity | Durability | Activation | Dimensional |
|---|---|---|---|---|---|---|---|---|
| Manufacturer | Grade | type | Shore A | conc. (w/w) | (0-10) | (0-10) | speed(0-10) | stability |

Thermoplastic elastomers Shore A-interval 35 to 80

| | | | | | | | | |
|---|---|---|---|---|---|---|---|---|
| HEXPOL TPE ™ | Mediprene ™ | TPE | 37 | 15.4% | 2 | 3 | 10 | 9 |
| | 500352M | | | | | | | |
| | | | | 25.1% | 6 | 3 | 9 | 7 |
| | | | | 35.2% | 10 | 4 | 9 | 6 |
| | | | | 40.1% | 10 | 4 | 10 | 5 |
| HEXPOL TPE ™ | Mediprene ™ | TPE | 46 | 25.1% | 6 | 5 | 7 | 8 |
| | 500452M | | | | | | | |
| | | | | 31.1% | 8 | 7 | 7 | 8 |
| | | | | 35.6% | 10 | 7 | 7 | 7 |
| | | | | 41.3% | 10 | 7 | 10 | 7 |
| | | | | 45.2% | 10 | 8 | 10 | 6 |
| Kraiburg ™ | TM5ADT ™ | TPE | 51 | 35.0% | 8 | 7 | 10 | 8 |
| | Thermolast ™ | | | | | | | |
| HEXPOL ™ TPE | Mediprene ™ TO | TPE | 57 | 29.2% | 4 | 2 | 5 | 9 |
| | 39555 | | | | | | | |
| HEXPOL ™ TPE | Mediprene ™ | TPE | 62 | 30.0% | 7 | 7 | 7 | 9 |
| | 500602M | | | | | | | |
| | | | | 41.2% | 8 | 77 | 7 | 9 |
| | | | | 50.7% | 9 | 8 | 10 | 8 |
| Kraiburg ™ | TM7MED ™ | TPE | 68 | 32.5% | 6 | 6 | 7 | 9 |
| | Thermolast ™ | | | | | | | |
| | | | | 35.0% | 7 | 7 | 7 | 9 |
| | | | | 40.0% | 7 | 7 | 7 | 8 |
| | | | | 50.0% | 8 | 8 | 10 | 7 |

Thermoplastic elastomers Shore A-interval 80 to 100

| | | | | | | | | |
|---|---|---|---|---|---|---|---|---|
| Dow ™ | Engage ™ 8401 | TPE | 84 | 70% | 4 | 6 | 3 | 6 |
| INEOS ™ | Styroflex ™ 2G66 | TPE | 84 | 71% | 3 | 6 | 2 | 6 |
| Dow ™ | Engage 8402 | TPE | 89 | 70% | 3 | 6 | 5 | 6 |
| TOPAS ™ AP | Elastomer ™ | TPE | 90 | 60% | 2 | 3 | 1 | 9 |
| | E-140 | | | | | | | |

TABLE 3
Thermoplastic polyurethanes Shore A-interval 70 to 80

| | | Material | Measured | Hydrophilic | Lubricity | Durability | Activation | Dimensional |
|---|---|---|---|---|---|---|---|---|
| Manufacturer | Grade | type | Shore A | conc. (w/w) | (0-10) | (0-10) | speed(0-10) | stability |
| BASF ™ | Elastollan | TPU | 72 | 41.1% | 6 | 7 | 7 | 10 |
| polyurethanes | 1170A ™ | | | | | | | |
| | | | | 48.0% | 8 | 8 | 7 | 9 |
| | | | | 51.0% | 9 | 9 | 8 | 9 |
| | | | | 60.0% | 9 | 10 | 10 | 6 |
| | | | | 64.6% | 10 | 10 | 10 | 4 |
| Lubrizol ™ | Estane ™ | TPU | 74 | 33.3% | 2 | 2 | 3 | 10 |
| | 58125 | | | | | | | |
| | | | | 37.5% | 5 | 4 | 5 | 9 |
| | | | | 44.5% | 8 | 7 | 7 | 8 |
| | | | | 49.9% | 9 | 7 | 8 | 7 |
| | | | | 57.3% | 9 | 8 | 10 | 3 |

TABLE 4
Thermoplastic polyurethanes Shore A-interval 80 to 100

| | | Material | Measured | Hydrophilic | Lubricity | Durability | Activation | Dimensional |
|---|---|---|---|---|---|---|---|---|
| Manufacturer | Grade | type | Shore A | conc. (w/w) | (0-10) | (0-10) | speed(0-10) | stability |
| Lubrizon ™ | Estane ™ | TPU | 86 | 20% | 1 | 2 | 1 | 9 |
| | 582701 | | | | | | | |
| | | | | 31% | 2 | 3 | 2 | 8 |
| | | | | 40% | 4 | 3 | 5 | 6 |
| | | | | 50% | 7 | 5 | 8 | 5 |
| | | | | 67% | 8 | 7 | 10 | 3 |
| | | | | 75% | 8 | 8 | 10 | 2 |
| BASF ™ | Elastollan ™ | TPU | 87 | 69% | 4 | 8 | 7 | 2 |
| polyurethanes | 1170A | | | | | | | |

TABLE 5
Thermoforming plastics Shore A-value out of scale (>100)

| | | Material | Measured | Hydrophilic | Lubricity | Durability | Activation | Dimensional |
|---|---|---|---|---|---|---|---|---|
| Manufacturer | Grade | type | Shore A | conc. (w/w) | (0-10) | (0-10) | Speed(0-10) | stability |
| Lyondellbasell | Purell ™ 2410T | PE | >100 | 51% | 1 | 0 | 3 | 3 |
| Lyondellbasell | Purell ™ HP671T | PPE | >100 | 50% | 1 | 1 | 3 | 4 |
| Nexeo ™ | Akulon ™ K222-D | PA-6 | >100 | 50% | 0 | 0 | 2 | 10 |
| Solutions | | | | | | | | |

### Optimum Molecular Mass of Plasticizer PEG for Compounding

From the compounding experiments it was concluded that, preferably, compounding of a selected matrix polymer with PEO should take place in the presence of a plasticizer selective for PEO. In general, as PEO's having molecular weights between 100 kD and 1,000 kD are relatively hard materials (Shore A 100 and up), compounding with a plasticizer selective for PEO helps lowering the hardness of the hydrophilic polymer component in the polymer compositions of the present interest.

And, while PEO can be plasticized with many materials, in the presently reported experiments only short chain PEO's (typically called polyethylene glycols (PEG) rather than polyethylene oxides (PEO)) were considered. The most relevant PEG-plasticizers are low molecular weight PEG's that are liquid at room temperature. PEG-200, PEG-300, PEG-400, and PEG-500 were tested and found suitable. Since PEO and PEG is the same material, PEO's and PEG's are chemically compatible at all concentrations, and the PEG-plasticizers are safe for compounding with the main hydrophilic PEO-polymers.

In conclusion, the main lubricious material should be PEO with MW from 200,000 and 500,000, preferably from 300,000 D to 450,000 D, more preferably 400,000 D; as plasticizer should be PEG with molecular weights from 200 D to 500 D, preferably from 300 D to 400 D, ranging in concentration from 10 w/w % to 20 w/w %. Optimal results were found in the with from 12.5 w/w % to 17.5 w/w %. Below 10% PEG and the material is similar in hardness to the pure PEO. For higher PEG concentration the compound material becomes softer.

### Experimental Conclusions and Discussion

Across the Tables 2-6, results for polymer blends of the invention having only suitability scores of 6 or above were found to be suitable for providing molded lubricious items. Experimentally, it was found that preferably all suitability scores should be 7 or above, with not more than a single suitability score of 6, most preferably without any single suitability score of 6.

The present experiments support the conclusion that the polymer blends of the invention are operative in the known manner according to the prior art, wherein the lubricious polymer at the device surface does not become fully lubricating before having been wet, even where the lubricious layer is lubricating before having been wet.

From the experiments it appears that a favorable balance between entanglement of the hydrophilic polymer and the matrix polymer can be achieved compared to the prior art and thus the dissolution of the hydrophilic polymer is retarded, but not stopped. However, as shown in the experiments, the polymer compositions of the invention permit maintenance of lubricity by permitting hydrophilic co-polymers held deeper in the matrix polymer to migrate to the surface of the polymer composition for maintaining lubricity and durability. However, over time the migration slows down due to the longer migration distance, and the lubrication thus decreases and eventually stops.

The present experiments have shown that migration to the surface is mainly controlled by three aspects of the polymer blends of the invention.
1) The concentration ratio of hydrophilic polymer to matrix polymer: Too low matrix polymer concentration permits more hydrophilic polymer to migrate to the surface, and there is little matrix polymer to retard migration. The result is strong lubrication and long duration, but the entire medical device is dissolved or deformed over time, whereas for high matrix polymer concentrations, there are less hydrophilic polymer to migrate to the surface, and there is much matrix polymer to retard migration. The result is little lubrication and short duration.
2) The hardness of the matrix polymer: For a hard matrix polymer (high Shore durometer hardness), entanglement between hydrophilic polymer and the matrix polymer is strong, and this significantly retards migration to the surface. The result is weak lubrication and short duration. For a soft matrix material (low Shore durometer hardness), entanglement between hydrophilic polymer and the matrix polymer is weaker, and this minimally retards migration to the surface. The result is strong lubrication and long duration.
3) The length of the hydrophilic polymer chain: Short hydrophilic polymer chain length lubricates the surface poorly, and the migration to the surface is fast. The result is little lubrication. Medium hydrophilic polymer chain length lubricates the surface well, and the migration to the surface is medium. Long hydrophilic polymer chain length lubricates the surface very well, but the migration to the surface is slow. The result is medium lubrication but short duration. Furthermore, very long polymer chain lengths are difficult or impossible to thermoform.

From this description it is apparent that a compromise between lubricity (degree of lubrication) and durability (duration of lubrication) is needed, as both a strong lubrication and a long duration could not be realized in the present experiments for a material that can be thermoformed and are dimensionally stable in use.

However, the present experiments surprisingly showed that a meaningful compromise between lubricity and durability can be achieved for:
a. Medium concentrations of hydrophilic polymer and matrix polymer.
b. Medium hardness of the matrix polymer.
c. Medium length of the hydrophilic polymer.

Some medical devices may require strong lubrication but for short time, while others require long time lubrication and medium lubrication is acceptable. Some variation over the medium concentrations, medium hardness, and medium polymer chain length, is thus needed for optimization for a given medical device application.

### Hardness Matching

Experimentally it was found that the hydrophilic polymer and the matrix polymer should have similar shore A hardness for mechanical compatibility and best results.

Since the relevant hydrophilic polymers are relatively hard (Shore A 100 and up), and relevant matrix polymers are softer (Shore A 50-70), the addition of a plasticizer for the hydrophilic polymer was found to be advantageous.

The present experiments have shown that while the hardness of the hydrophilic polymer and the matrix polymer should be similar, the Shore A hardness of the matrix polymer and hydrophilic polymer does not need to be identical, but the difference in Shore A hardness should not be more than 40. Matrix polymer harder than Shore A 90 will not work.

### Concentration of Hydrophilic Polymer Versus Shore a Hardness of the Matrix Polymer

Experimentally, it was found that the optimum concentration of hydrophilic polymer depends on the Shore A hardness of the matrix material. The softer the matrix material the less hydrophilic polymer is required to obtain a relevant combination of lubricity and durability.
Shore A 90-80% hydrophilic polymer (test device deforms/dissolves during use) Shore A 80-70% hydrophilic polymer (test device deforms during use)
Shore A 70-50% hydrophilic polymer Shore A 60-35% hydrophilic polymer Shore A 50-25% hydrophilic polymer Shore A 40-15% hydrophilic polymer (test device has low lubricity)

These are average results obtained from many tests, such as reported in Tables 2 to 6, using Estane™ and Elastollan™ thermoplastic polyurethanes, Topas™ E-140 elastomer, and Kreiburg™ thermoplastic elastomers all of different Shore A hardness. The hydrophilic polymer was PEO-400.000 plasticized with 15% PEG-400.

### Practical Workspace

From these results the present inventor concludes that practical lubrication results relevant for medical devices, are obtained when the matrix polymer has Shore A hardness between 45 and 75 and the polymer blend comprises between 20% and 65% hydrophilic polymer.

This practical workspace is for relatively soft matrix polymers, with relatively high loading of hydrophilic polymer. As most medical devices are molded in materials harder than Shore A 75, and additives to polymers are generally in low concentration (1%-10%), more than 20% plasticizer is very rare, the present inventor has found that commonly used materials and intuitive additive concentrations are outside the range suitable for obtaining a lubricating polymer blend according to the present invention.

With the present invention it is possible to make compound materials that can be molded, are dimensionally stable and give good lubrication and durability when contacting water.

### Closing Comments

Although the present invention has been described in detail for purpose of illustration, it is understood that such detail is solely for that purpose, and variations can be made therein by those skilled in the art in practicing the claimed subject matter, from a study of the drawings, the disclosure, and the appended claims. The term “comprising” as used in the claims does not exclude other elements or steps. The indefinite article “a” or “an” as used in the claims does not exclude a plurality.

1. A polymer blend comprising a matrix polymer and a hydrophilic polymer composition comprising polyethylene-oxide; said matrix polymer selected from a thermoplastic elastomer (TPE) or a thermoplastic polyurethane (TPU) matrix polymer presenting a flat-surface Shore A hardness from 40 to 75 according to ISO 48-4:2018;
said polymer blend comprising, by total polymer blend weight, from 20 w/w % to 65 w/w % of said hydrophilic polymer composition with the balance matrix polymer;
said hydrophilic polymer composition comprising, by weight of said hydrophilic polymer composition, from 5 w/w % to 30 w/w % of a PEG-plasticizer having a molecular weight from 200 D to 600 D, and polyethyleneoxide having a molecular weight from 100,000 D to 750,000 D as balance.

2. The polymer blend according to claim 1, wherein said flat-surface Shore A hardness is from 45 to 75 according to ISO 48-4:2018.

3. The polymer blend according to claim 1, wherein said hydrophilic polymer composition comprises polyethyleneoxide having a molecular weight from 200,000 D to 500,000 D.

4. The polymer blend according to claim 1, wherein said polyethyleneoxide has a molecular weight of from 350,000 D to 450,000.

5. The polymer blend according to claim 1, wherein said PEG-plasticizer has a molecular weight from 300 D to 500 D.

6. The polymer blend according to claim 1, wherein said PEG-plasticizer has a molecular weight of from 350 D to 450 D.

7. The polymer blend according to claim 1, wherein said hydrophilic polymer composition comprises, by weight of said hydrophilic polymer composition, from 10 w/w % to 20 w/w % of said PEG-plasticizer.

8. The polymer blend according to claim 1, wherein said hydrophilic polymer composition comprises, by weight of said hydrophilic polymer composition, 15 w/w % of said PEG-plasticizer

9. (canceled)

10. A medical article comprising a polymer blend according to claim 1, presenting when wet at least one lubricious surface made from said polymer blend.
